(19) United States
(12) Patent Application Publication
(10) Pub. No.: US 2025/0232088 A1
(43) Pub. Date: Jul. 17, 2025

(54) METHOD FOR PREDICTING MICROSCOPIC HOLE OF ALUMINUM ALLOY PRODUCT AND IMPACT ON MACROSCOPIC SERVICE PROPERTY

(71) Applicant: CITIC Dicastal Co., Ltd., Qinhuangdao City (CN)

(72) Inventors: Yuling Lang, Qinhuangdao City (CN); Xiaoying Ma, Qinhuangdao City (CN); Decai Kong, Qinhuangdao City (CN); Haibo Qiao, Qinhuangdao City (CN); Zhongyao Li, Qinhuangdao City (CN); Zhihua Zhu, Qinhuangdao City (CN); Shiwen Xu, Qinhuangdao City (CN); Xiang Ci, Qinhuangdao City (CN); Wenbo Wang, Qinhuangdao City (CN); Jian Gao, Qinhuangdao City (CN)

(73) Assignee: CITIC Dicastal Co., Ltd., Qinhuangdao City (CN)

(21) Appl. No.: 19/096,634

(22) Filed: Mar. 31, 2025

(30) Foreign Application Priority Data
Apr. 3, 2024 (CN) CN202410401940.5

(51) Int. Cl. G06F 30/23; G06F 119/02

(52) U.S. Cl. CPC G06F 30/23; G06F 2119/02

(57) ABSTRACT

A method for predicting microscopic holes of an aluminum alloy product and impact on macroscopic service properties includes: casting simulation, i.e., obtaining a casting simulation finite element mesh by dividing, and using casting simulation software to simulate a solidification process of a casting under corresponding process conditions to obtain macroshrinkages of the casting and physical information of each node on the mesh; cellular automata simulation, i.e., simulating microstructure growth by using a cellular automata model to obtain a secondary dendrite arm spacing (SDAS) value at each node of the casting simulation finite element mesh, and the morphology and size of microscopic holes including microshrinkages and microscopic blowholes; mechanical property simulation, and mapping and inputting mesh information of the casting simulation finite element mesh into the mechanical and fatigue property simulation finite element mesh to obtain a mechanical property simulation result; and fatigue property simulation.

### FIELD OF THE INVENTION

The present disclosure relates to the technical field of material computing, in particular to a method for predicting microscopic holes of an aluminum alloy product and impact on macroscopic service properties.

### BACKGROUND OF THE INVENTION

Cast aluminum alloys have become one of indispensable materials in modern manufacturing by virtue of their light weight, high strength, good formability, and wide application in multiple industrial fields. In the context of lightweight, by using the cast aluminum alloys, automobile manufacturers can reduce the vehicle weight, improve the fuel efficiency, and reduce emissions; the aerospace field has extremely high requirements for light weight, strength and corrosion resistance of materials, and the cast aluminum alloys are ideal materials for meeting these requirements. Many key components such as an aircraft structure and an engine part are manufactured by using the cast aluminum alloys to improve the flight efficiency and performance. In conclusion, the advantages of the cast aluminum alloys in terms of improving product properties, reducing energy consumption, driving the development of the lightweight technology, etc., make their positions in the industry constantly consolidated and improved.

Currently, in the production simulation of aluminum alloy castings, casting simulation software mostly considers macroshrinkages generated in the solidification process and cannot consider microscopic holes, and a cellular automata model can only simulate microstructure growth in micron- and millimeter-scale spaces in one computation, and cannot realize prediction of microscopic holes of the castings as a whole.

When performing various types of simulations, there are great problems in data interaction with each other due to different software used. The appearance of holes has a great influence on the mechanical properties and fatigue life of aluminum alloys. Currently, the casting simulation software cannot achieve mechanical property simulation, neither mechanical property simulation nor fatigue property simulation software have the ability to take into account shrinkages, and the results obtained by setting homogeneous materials are quite different from a real situation. Therefore, it is particularly urgent to complete the mapping of the shrinkage information computed by the casting simulation software and a cellular automata to a mechanical property and fatigue property simulation software to achieve calculations that take into account the influence of shrinkage information on the properties of the aluminum alloys.

Existing patent documents:
In patent document 1 (JP2021053662A), the step of predicting shrinkages is to obtain virtual inscribed spheres inscribed to the surface of a casting at various points inside the casting. The distance from an inner point of a larger virtual inscribed sphere to the surface of the casting is relatively large, indicating that liquid metal is difficult to cool at the inner point. Otherwise, the distance from an inner point of a smaller virtual inscribed sphere to the surface of the casting is smaller, indicating that the liquid metal is relatively easy to cool at the inner point;
In patent document 2 (JP2021094562A), the step of predicting shrinkages includes establishing a machine learning model, learning a relationship between blowhole occurrence probability distributions by actual measurement, and after training, predicting a distribution of the shrinkages based on three-dimensional voxel data;
In patent document 3 (JP7256448B2), the step of predicting shrinkages is to predict the occurrence of shrinkages by casting analysis in which the solidification time is calculated according to the temperature change of molten metal; and

In contrast, the above three patent documents focus on the prediction of shrinkages on a macroscopic scale and still have shortcomings in the quantitative analysis of microscopic holes.

In view of this, the method for predicting microscopic holes of an aluminum alloy product and impact on macroscopic service properties provided in the present disclosure aims to achieve simultaneous cross-scale prediction of locations of the macroshrinkages and the morphology volume of microscopic holes of a casting as a whole, and can also simulate secondary dendrite arm growth of the casting as a whole, providing a basis for mechanical property simulation and fatigue property simulation.

### SUMMARY OF THE INVENTION

An object of the present disclosure is to provide a method for predicting microscopic holes of an aluminum alloy product and impact on macroscopic service properties, so as to solve the problems set forth in the background.

According to one aspect of the present disclosure, provided is a method for predicting microscopic holes of an aluminum alloy product and impact on macroscopic service properties, including the steps of: S1: casting simulation, i.e., dividing finite element meshes of a casting to obtain a casting simulation finite element mesh, and using casting simulation software to simulate a solidification process of the casting under corresponding process conditions to obtain macroshrinkages of the casting and physical information of each node on the casting simulation finite element mesh; S2: cellular automata simulation, i.e., simulating microstructure growth by using a cellular automata model, wherein microstructure growth simulation is performed by using the physical information of each node on the casting simulation finite element mesh as an input to the cellular automata model to obtain a secondary dendrite arm spacing (SDAS) value at each node of the casting simulation finite element mesh, and the morphology and size of microscopic holes including microshrinkages and microscopic blowholes; S3: mechanical property simulation, i.e., dividing a mechanical and fatigue property simulation finite element mesh of the casting by using mechanical property simulation software, mapping and inputting mesh information of the casting simulation finite element mesh into the mechanical and fatigue property simulation finite element mesh, and performing mechanical property simulation according to use conditions by taking into account the influence of the macroshrinkages, the microscopic holes and the SDAS values on the mechanical properties of an aluminum alloy to obtain a mechanical property simulation result; and S4: fatigue property simulation, i.e., performing fatigue property simulation by using the mechanical property simulation result, and taking into account the influence of the macroshrinkages, the microscopic holes and the SDAS values on the fatigue properties of the aluminum alloy to obtain a fatigue property simulation result of the casting under rated working conditions.

Preferably, the step S1 further includes: S101: establishing a casting simulation model, including: setting boundary conditions and simulation parameters according to casting process conditions; and S102: outputting an average cooling rate of all nodes on the casting simulation finite element mesh during solidification by secondary development to the casting simulation software, inputting node information including numbers and coordinates into a casting simulation result file, and inputting into a database.

Preferably, the step S2 further includes: S201: using the cellular automata model to simulate microstructure growth at all nodes on the casting simulation finite element mesh with the average cooling rate as an input to obtain a cellular automata simulation result including morphology and size data of the microscopic holes, and the SDAS values; and S202: inputting the cellular automata simulation result into a corresponding node of the database for storage, wherein a size of each microscopic hole is described by an equivalent diameter of the microscopic hole at the node and a maximum length of the microscopic hole at the node.

Preferably, the step S3 further includes: S301: performing mapping input on the casting simulation result file and a cellular automata result file, and completing material assignment of a mechanical property simulation model at a mesh level; S302: completing application of constraints and loads according to the use requirements of the casting, and performing mechanical property simulation; and S303: obtaining a mechanical property simulation result file, and generating and saving a simulation result report.

Preferably, the step S4 further includes: S401: performing mapping input on the mechanical property simulation result file, the casting simulation result file and the cellular automata result file, and completing material assignment of a fatigue property simulation model at a mesh node level; S402: performing fatigue property simulation according to national standards or use conditions of the casting; and S403: obtaining a fatigue property simulation result file, and generating and saving a simulation result report.

Preferably, in S102, the database takes coordinates of casting finite element mesh nodes as a primary key, and fields in each row include: node coordinates, a node number, the average cooling rate at the nodes, a macroshrinkage porosity at the node, the equivalent diameter of the microscopic hole at the node, the maximum length of the microscopic hole at the node, and the SDAS value at the node, wherein the node coordinates, the node number, the average cooling rate at the nodes, and the macroshrinkage porosity at the node are from the casting simulation; and the equivalent diameter of the microscopic hole at the node, the maximum length of the microscopic hole at the node, and the SDAS value at the node are from the cellular automata simulation.

Preferably, a solidification shrinkage prediction model for predicting microshrinkages in the microscopic holes and a dendrite nucleation and growth model with supercooling as a driving force are included in the cellular automata model, the average cooling rate is used as an input to the dendrite nucleation and growth model, solid fraction information for each cell provided by the dendrite nucleation and growth model is input into the solidification shrinkage prediction model, and a pressure drop required to form holes is calculated to predict solidification shrinkages.

Preferably, the mapping input is performed by a mesh information mapping algorithm for: automatically reading mesh information output by the casting simulation software; automatically reading macroshrinkage information output by the casting simulation software; automatically reading microscopic hole information and the SDAS value output by the cellular automata simulation; automatically implementing mapping of the mesh information; automatically implementing influence analysis of the macroshrinkage and microscopic hole information and the SDAS value by the mechanical property simulation software; and automatically performing material assignment of the mechanical property simulation software at a mesh level.

Preferably, the influence of the macroshrinkages, the microscopic holes and the SDAS values on the mechanical properties of the aluminum alloy is expressed as a mathematical relationship between the yield strength, fracture strain, and Young's modulus of the aluminum alloy, and the macroshrinkages, the microscopic holes and the SDAS values, and/or the influence of the macroshrinkages, the microscopic holes and the SDAS values on fatigue property parameters is expressed as a mathematical relationship between the tensile strength and fatigue strength of the aluminum alloy, and the macroshrinkages, the microscopic holes and the SDAS values.

According to another aspect of the present disclosure, provided is a recording medium, having recorded thereon a computer program configured to cause a computer to implement any one of the above methods.

Compared with the prior art, the beneficial effects of the present disclosure are as follows:
1. The method of the present disclosure can accurately predict hole location and volume information of aluminum alloy castings on a macroscopic scale, and accurately predict the morphology and size of microscopic holes (including shrinkages and blowholes) of aluminum alloy castings on a microscopic scale.
2. The method of the present disclosure can realize the prediction of the influence of holes and SDAS values on the mechanical properties and fatigue life of aluminum alloy castings, and can be used to guide the improvement of the casting process and precisely perform the simulation research and experimental exploration of the strength and fatigue properties of the corresponding aluminum alloy cast products.

### BRIEF DESCRIPTION OF DRAWINGS

FIG. 1 is an exemplary flow diagram of a method for predicting microscopic holes of an aluminum alloy product and impact on macroscopic service properties according to the present disclosure;

FIG. 2 is a flow chart of a method according to an embodiment of the present disclosure;

FIG. 3 shows the macroscopic shrinkage results of a casting obtained in casting simulation software, as well as the simulation results of microscopic holes and secondary dendrite arms on one of the nodes according to an embodiment of the present disclosure;

FIG. 4 is a schematic diagram of the results obtained after material assignment to a casting in mechanical property simulation software under the condition that the influence of the macroshrinkages, the microscopic holes and the SDAS values on parameters such as Young's modulus and yield strength is considered at a mesh level according to an embodiment of the present disclosure; and

FIG. 5 is a schematic flow diagram of a prediction method according to the present disclosure.

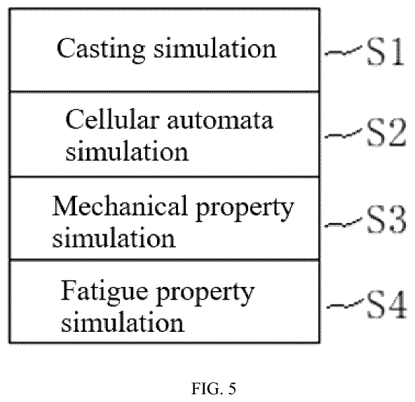

### DETAILED DESCRIPTION OF THE INVENTION

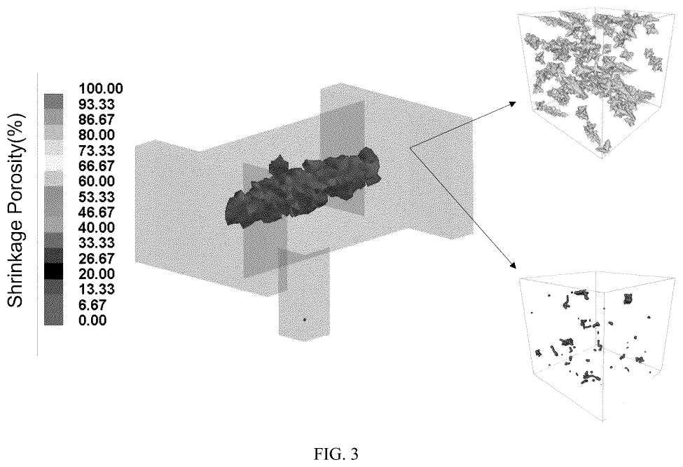

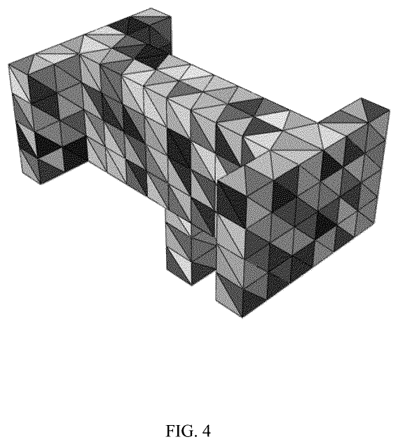

To achieve the above object, the present disclosure provides the following technical solution, including the steps of:
S1: casting simulation, i.e., dividing finite element meshes of a casting to obtain a casting simulation finite element mesh, and using casting simulation software to simulate a solidification process of the casting under corresponding process conditions to obtain macroshrinkages of the casting and physical information of each node on the mesh (see FIG. 3);
S2: cellular automata simulation, i.e., simulating microstructure growth, wherein microstructure growth simulation is performed by using the physical information of each node on the casting simulation finite element mesh as an input to a cellular automata model to obtain a secondary dendrite arm spacing (SDAS) value at each node of the casting simulation finite element mesh, and the morphology and size of microscopic holes (including microshrinkages and microscopic blowholes);
S3: mechanical property simulation, i.e., dividing a mechanical and fatigue property simulation finite element mesh of the casting (i.e., the mechanical property simulation uses the same finite element mesh as fatigue property simulation), using a mesh mapping algorithm to map mesh information of the casting simulation finite element mesh into the mechanical and fatigue property simulation finite element mesh, and performing mechanical property simulation according to use conditions by taking into account the influence of the macroshrinkages, the microscopic holes and the SDAS values on the mechanical properties of the aluminum alloy product to obtain parameters such as stress strain (see FIGS. 4); and
S4: fatigue property simulation, i.e., performing fatigue property simulation by using a mechanical property simulation result obtained in the step S3, and taking into account the influence of the macroshrinkages, the microscopic holes and the SDAS values on the fatigue properties of the aluminum alloy product according to national standards or use conditions of the casting to obtain a fatigue property simulation result of a part under rated working conditions, for example, parameters such as the service life.

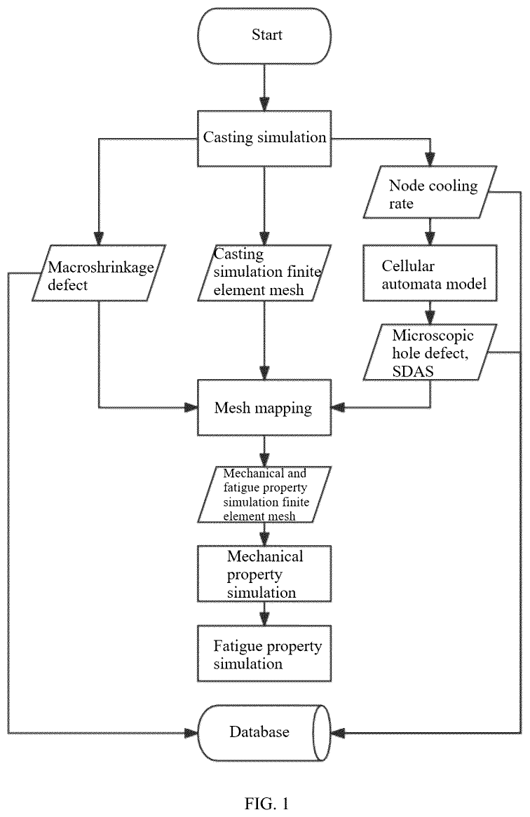

Further, the step S1 specifically further includes:
S101: establishing a casting simulation model, including: setting boundary conditions and simulation parameters according to casting process conditions; and
S102: outputting an average cooling rate (also called a node cooling rate, see FIG. 1) of all nodes on the casting simulation finite element mesh during solidification by secondary development of the casting simulation software, while inputting coordinates and numbers of the nodes into a casting simulation result file and then into a database.

Further, the step S2 specifically further includes:
S201: using the cellular automata model to simulate microstructure growth at all nodes on the casting simulation finite element mesh with the average cooling rate of the casting simulation in S102 as an input to obtain morphology and size data of the microscopic holes, and the SDAS values; and
S202: inputting a cellular automata simulation result in S201 into a corresponding node of the database for storage, wherein an equivalent diameter of each microscopic hole at the node, and a maximum length of the microscopic hole at the node which are used to describe a size of the microscopic hole, and the SDAS values at the nodes are included.

Further, the step S3 specifically further includes:
S301: performing mapping input on the casting simulation result file and a cellular automata result file, and completing material assignment of a mechanical property simulation model at a mesh level;
S302: completing an application of constraints and loads according to the use requirements of the casting, and performing mechanical property simulation; and
S303: obtaining a mechanical property simulation result file, and generating and saving a mechanical property simulation result report.

Further, the step S4 specifically further includes:
S401: performing mapping input on the mechanical property simulation result file, the casting simulation result file and the cellular automata result file, and completing material assignment of a fatigue property simulation model at a mesh node level;
S402: performing fatigue property simulation according to national standards or use conditions of the casting; and
S403: obtaining a fatigue property simulation result file, and generating and saving a fatigue property simulation result report.

Further, in S102, the database takes coordinates of casting simulation finite element mesh nodes as a primary key, and fields in each row include: node coordinates, a node number, the average cooling rate at the nodes, a macroshrinkage porosity at the node (see FIG. 3), the equivalent diameter of the microscopic hole at the node, the maximum length of the microscopic hole at the node, and the SDAS value at the node.

Further, in S102, the node coordinates, the node number, the average cooling rate at the nodes, and the macroshrinkage porosity at the node are from the casting simulation; and the equivalent diameter of the microscopic hole at the node, the maximum length of the microscopic hole at the node, and the SDAS value at the node are from the cellular automata simulation.

A solidification shrinkage prediction model for predicting microshrinkages in the microscopic holes and a dendrite nucleation and growth model with supercooling as a driving force are included in the cellular automata model. The average cooling rate of the casting simulation is used as an input to the dendrite nucleation and growth model, solid fraction information for each cell provided by the dendrite nucleation and growth model is input into the solidification shrinkage prediction model, and a pressure drop required to form holes is calculated to achieve the purpose of predicting solidification shrinkages.

Further, accurate mapping between both (i.e., the casting simulation software and the cellular automata model) and mechanical property simulation software is achieved by an autonomously developed mapping algorithm.

Further, a code that can be called in the background is written in a python language to achieve information transfer between two different finite element meshes, including coordinate information at the nodes and the meshes, hole information, and information of SDAS values.

Further, the mesh information mapping algorithm has the following functions:

automatically reading mesh information output by the casting simulation software;

automatically reading macroshrinkage information output by the casting simulation software;

automatically reading microscopic hole information and the SDAS values output by a cellular automata;

automatically implementing mapping of the mesh information;

automatically implementing influence analysis of the macroshrinkage and microscopic hole information and the SDAS values by the mechanical property simulation software; and

automatically performing material assignment of the mechanical property simulation software at a mesh level.

Further, the specific effects of the macroshrinkages, the microscopic holes and the SDAS values on mechanical property parameters are explored, specifically: through a series of experiments and investigations, a general mathematical relationship between the yield strength, fracture strain, and Young's modulus of the aluminum alloy, and the macroshrinkages, the microscopic holes and the SDAS values is obtained.

Further, accurate mapping between both (i.e., the casting simulation software and the cellular automata model) and fatigue property simulation software is achieved by an autonomously developed mapping algorithm.

Further, a code that can be called in the background is written in a python language to achieve information transfer between two different finite element meshes, including coordinate information at the nodes and the meshes, hole information, and information of SDAS values.

Further, the specific effects of the macroshrinkages, the microscopic holes and the SDAS values on fatigue property parameters are explored, specifically: through a series of experiments and investigations, a general mathematical relationship between the tensile strength and fatigue strength of the aluminum alloy, and the macroshrinkages, the microscopic holes and the SDAS values is obtained.

### Embodiment 1

FIG. 1 shows a schematic flow diagram of a method for predicting microscopic holes of an aluminum alloy product and impact on macroscopic service properties according to an embodiment of the present disclosure. As shown in FIG. 1, the method includes:
S101′: dividing a casting simulation finite element mesh for an aluminum alloy casting, setting boundary conditions and simulation parameters according to casting process conditions, establishing a casting simulation model, and performing S102′ during simulation;
S102′: executing a secondary development program by casting simulation software, reading node numbers, node coordinates, mesh numbers, and mesh vertex numbers of the casting simulation finite element mesh at the beginning of the simulation, monitoring temperature fields of all finite element mesh nodes on a casting during the simulation, and after calculating an average cooling rate, outputting the above calculation result into a casting simulation result file for storage, and then inputting into a database;
S201′: reading, by a cellular automata model, the average cooling rate obtained in S102′, calculating dendrite growth and hole formation in microscopic regions at the positions of the nodes of the casting simulation finite element mesh, and realizing prediction of microscopic holes of the casting as a whole, and inputting simulation results of an equivalent diameter of the microscopic holes, a maximum length of the microscopic holes, and SDAS values into a database after the end of cellular automata simulation;
S301′: dividing, by mechanical property simulation software, a mechanical and fatigue property simulation finite element mesh of the casting, using a mesh mapping algorithm to map mesh information of the casting simulation finite element mesh into the mechanical and fatigue property simulation finite element mesh, and performing mechanical property simulation according to use conditions by taking into account the influence of macroshrinkages, the microscopic holes and the SDAS values on the mechanical properties of an aluminum alloy to obtain parameters such as stress strain; and
S401′: performing fatigue property simulation by fatigue property simulation software by using a mechanical property simulation result obtained in the step S301′, using the mechanical and fatigue property simulation finite element mesh already containing the mesh information of the casting simulation finite element mesh, and taking into account the influence of the macroshrinkages, the microscopic holes and the SDAS values on the fatigue properties of the aluminum alloy according to national standards or use conditions of the casting to obtain parameters such as the service life of the casting under rated working conditions.

Further, considering the influence of the macroshrinkages, the microscopic holes and the SDAS values on the fatigue properties of the aluminum alloy, an expression for the influence of the macroshrinkages, the microscopic holes and the SDAS values on the properties is fitted by an XCT experiment combined with a tensile test, and accurately mapped into each mesh.

### Embodiment 2

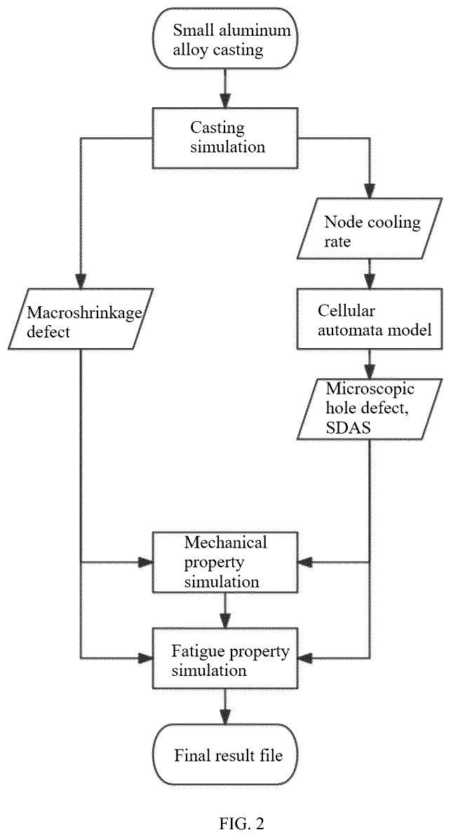

Optionally, as one embodiment of the present disclosure, as shown in FIG. 2, which is a specific embodiment of a method for predicting microscopic holes of an aluminum alloy product and impact on macroscopic service properties according to the present disclosure, taking a small aluminum alloy casting as an example, through the method for predicting microscopic holes of an aluminum alloy product and impact on macroscopic service properties of the present disclosure, macroshrinkage defects and microscopic hole defects inside the casting are predicted, and mechanical properties and fatigue properties of the casting are predicted in consideration of the defects of the casting. Specific steps include:
S101″: a casting simulation finite element mesh is divided for the small aluminum alloy casting, wherein a division result is as follows: 79935 tetrahedral meshes, and 17020 nodes on the casting. The establishment of a casting simulation model continues to be completed;
S102″: a secondary development program is automatically called during simulation run, firstly, numbers and coordinates of all the nodes on the casting simulation finite element mesh, and labels and vertex numbers of all the tetrahedral meshes are read, and stored in a corresponding casting simulation result file. Then, an average cooling rate of all the nodes is calculated during the simulation run, and stored in the corresponding casting simulation result file, and after the simulation is ended, the numbers, the coordinates and the average cooling rate of the 17020 nodes are read from the casting simulation result file, and input into a database for storage;
S201″: a cellular automata model starts simulating a solidification process on a microscopic scale by reading the average cooling rate of the 17020 nodes from the casting simulation result file in S102″ or from the database. And microscopic hole information at corresponding positions on all the nodes is calculated, including the equivalent diameter and the maximum length which are used for describing the size of the microscopic holes, and SDAS values at the nodes;
S202″: the cellular automata model inputs data obtained after the simulation in S201″ into rows of the corresponding nodes of the database;
S301″: a mechanical and fatigue property simulation finite element mesh is divided for the small aluminum alloy casting, wherein a division result is as follows: 1006 tetrahedral meshes, and 1847 nodes on the casting. Mapping input is performed on the casting simulation result file and a cellular automata result file, and considering the influence of macroshrinkages, microscopic holes and SDAS values on the mechanical properties of an aluminum alloy, mainly considering the setting of parameters such as Young's modulus and yield strength, material assignment of a mechanical property simulation model at a mesh level is completed;
S302″: an application of constraints and loads is completed according to the use requirements of the small aluminum alloy casting, and mechanical property simulation is performed to obtain parameters such as stress strain;
S303″: a mechanical property simulation result file is obtained, and a mechanical property simulation result report is generated and saved.
S401″: mapping input is performed on the mechanical property simulation result file, the casting simulation result file and the cellular automata result file, and considering the influence of the macroshrinkages, the microscopic holes and the SDAS values on the fatigue properties of the aluminum alloy, mainly considering the setting of parameters such as tensile strength and fatigue strength, material assignment of a model at a mesh node level is completed;
S402″: fatigue property simulation is performed by selecting a suitable fatigue life calculation method according to national standards or use conditions of the casting to obtain prediction of the service life of different parts of a part; and
S403″: a fatigue property simulation result file is obtained, and a fatigue property simulation result report is generated and saved.

The method provided in the above embodiment realizes cross-scale prediction of hole defects in aluminum alloy castings, including macroscale prediction of solidification shrinkages, and microscale prediction of secondary dendrite arm growth, blowholes, and solidification shrinkages of the casting as a whole.

Further, in the method provided in the above embodiment, by the mesh mapping algorithm, considering the influence of the macroshrinkages, the microscopic holes and the SDAS values on the mechanical and fatigue properties of aluminum alloys, mainly considering the setting of parameters such as Young's modulus, yield strength, tensile strength, fracture strain, and fatigue strength, and coupling the influence of hole information and the SDAS values into the mechanical property simulation and the fatigue property simulation of the aluminum alloy castings, the prediction of the mechanical properties and fatigue properties of the aluminum alloy castings is achieved by considering hole defects.

Compared with the prior art in which a cellular automata only realizes computation in micron- and millimeter-scale spaces in one computation, in the step S2 of the present disclosure, the computation of the cellular automata is performed on nodes of each finite element mesh, thereby achieving the purpose of computing the microstructure of the entire casting.

1. A method for predicting microscopic holes of an aluminum alloy product and impact on macroscopic service properties, comprising the steps of:
S1: casting simulation, i.e., dividing finite element meshes of a casting to obtain a casting simulation finite element mesh, and using casting simulation software to simulate a solidification process of the casting under corresponding process conditions to obtain macroshrinkages of the casting and physical information of each node on the casting simulation finite element mesh;
S2: cellular automata simulation, i.e., simulating microstructure growth by using a cellular automata model, wherein microstructure growth simulation is performed by using the physical information of each node on the casting simulation finite element mesh as an input to the cellular automata model to obtain a secondary dendrite arm spacing (SDAS) value at each node of the casting simulation finite element mesh, and the morphology and size of microscopic holes including microshrinkages and microscopic blowholes;
S3: mechanical property simulation, i.e., dividing a mechanical and fatigue property simulation finite element mesh of the casting by using mechanical property simulation software, mapping and inputting mesh information of the casting simulation finite element mesh into the mechanical and fatigue property simulation finite element mesh, and performing mechanical property simulation according to use conditions by taking into account the influence of the macroshrinkages, the microscopic holes and the SDAS values on the mechanical properties of an aluminum alloy to obtain a mechanical property simulation result; and
S4: fatigue property simulation, i.e., performing fatigue property simulation by using the mechanical property simulation result and taking into account the influence of the macroshrinkages, the microscopic holes and the SDAS values on the fatigue properties of the aluminum alloy to obtain a fatigue property simulation result of the casting under rated working conditions.

2. The method for predicting microscopic holes of an aluminum alloy product and impact on macroscopic service properties according to claim 1, wherein the step S1 further comprises:
S101: establishing a casting simulation model, comprising: setting boundary conditions and simulation parameters according to casting process conditions; and
S102: outputting an average cooling rate of all nodes on the casting simulation finite element mesh during solidification by secondary development to the casting simulation software, inputting node information comprising numbers and coordinates into a casting simulation result file, and inputting into a database.

3. The method for predicting microscopic holes of an aluminum alloy product and impact on macroscopic service properties according to claim 2, wherein the step S2 further comprises:
S201: using the cellular automata model to simulate microstructure growth at all nodes on the casting simulation finite element mesh with the average cooling rate as an input to obtain a cellular automata simulation result comprising morphology and size data of the microscopic holes, and the SDAS values; and
S202: inputting the cellular automata simulation result into a corresponding node of the database for storage, wherein a size of each microscopic hole is described by an equivalent diameter of the microscopic hole at the node and a maximum length of the microscopic hole at the node.

4. The method for predicting microscopic holes of an aluminum alloy product and impact on macroscopic service properties according to claim 3, wherein the step S3 further comprises:
S301: performing mapping input on the casting simulation result file and a cellular automata result file, and completing material assignment of a mechanical property simulation model at a mesh level;
S302: completing an application of constraints and loads according to the use requirements of the casting, and performing mechanical property simulation; and
S303: obtaining a mechanical property simulation result file, and generating and saving a simulation result report.

5. The method for predicting microscopic holes of an aluminum alloy product and impact on macroscopic service properties according to claim 4, wherein the step S4 further comprises:
S401: performing mapping input on the mechanical property simulation result file, the casting simulation result file and the cellular automata result file, and completing material assignment of a fatigue property simulation model at a mesh node level;
S402: performing fatigue property simulation according to national standards or use conditions of the casting; and
S403: obtaining a fatigue property simulation result file, and generating and saving a simulation result report.

6. The method for predicting microscopic holes of an aluminum alloy product and impact on macroscopic service properties according to claim 5, wherein in S102, the database takes coordinates of casting finite element mesh nodes as a primary key, and fields in each row comprise: node coordinates, a node number, the average cooling rate at the nodes, a macroshrinkage porosity at the node, the equivalent diameter of the microscopic hole at the node, the maximum length of the microscopic hole at the node, and the SDAS value at the node, wherein the node coordinates, the node number, the average cooling rate at the nodes, and the macroshrinkage porosity at the node are from the casting simulation; and the equivalent diameter of the microscopic hole at the node, the maximum length of the microscopic hole at the node, and the SDAS value at the node are from the cellular automata simulation.

7. The method for predicting microscopic holes of an aluminum alloy product and impact on macroscopic service properties according to claim 1, wherein a solidification shrinkage prediction model for predicting microshrinkages in the microscopic holes and a dendrite nucleation and growth model with supercooling as a driving force are comprised in the cellular automata model, the average cooling rate is used as an input to the dendrite nucleation and growth model, solid fraction information for each cell provided by the dendrite nucleation and growth model is input into the solidification shrinkage prediction model, and a pressure drop required to form holes is calculated to predict solidification shrinkages.

8. The method for predicting microscopic holes of an aluminum alloy product and impact on macroscopic service properties according to claim 5, wherein the mapping input is performed by a mesh information mapping algorithm for: automatically reading mesh information output by the casting simulation software; automatically reading macroshrinkage information output by the casting simulation software; automatically reading microscopic hole information and the SDAS values output by the cellular automata simulation; automatically implementing mapping of the mesh information; automatically implementing influence analysis of the macroshrinkage and microscopic hole information and the SDAS values by the mechanical property simulation software; and automatically performing material assignment of the mechanical property simulation software at a mesh level.

9. The method for predicting microscopic holes of an aluminum alloy product and impact on macroscopic service properties according to claim 1, wherein the influence of the macroshrinkages, the microscopic holes and the SDAS values on the mechanical properties of the aluminum alloy is expressed as a mathematical relationship between the yield strength, fracture strain, and Young's modulus of the aluminum alloy, and the macroshrinkages, the microscopic holes and the SDAS values, and/or the influence of the macroshrinkages, the microscopic holes and the SDAS values on fatigue property parameters is expressed as a mathematical relationship between the tensile strength and fatigue strength of the aluminum alloy, and the macroshrinkages, the microscopic holes and the SDAS values.

10. A recording medium, having recorded thereon a computer program configured to cause a computer to implement the method according to claim 1.
